SEQUENCE LISTING (10 sequences)

SEQ ID NO: 1 - DNA, 112 bp, Bacteriophage M13
tcgctggatgtgtctgcggcgttttatcatcttcctctgcatcctgctgctatgcctcatcttcttgttggttcttctggactatcaaggtatgttgcccgtttgtcctcta

SEQ ID NO: 2 - DNA, 27 bp, Artificial Sequence
  primer
tcgctggatgtgtctgcggcgttttat

SEQ ID NO: 3 - DNA, 24 bp, Artificial Sequence
  primer
ggtatgttgcccgtttgtcctcta

SEQ ID NO: 4 - DNA, 29 bp, Artificial Sequence
  beacon
cctgctgctatgcctcatcttcttgttgg

SEQ ID NO: 5 - DNA, 239 bp, Homo sapiens
gctgtgtttgcgtctctcccaggaatcatctttaccagatctcaaaaagaaggtcttcattacacctgcagctctcattttccatacagtcagtatcaattctggaagaatttccagacattaaagatagtcatcttggggctggtcctgccgctgcttgtcatggtcatctgctactcgggaatcctaaaaactctgcttcggtgtcgaaatgagaagaagaggcacagggctgtgag

SEQ ID NO: 6 - DNA, 24 bp, Artificial Sequence
  primer
gctgtgtttgcgtctctcccagga

SEQ ID NO: 7 - DNA, 24 bp, Artificial Sequence
  primer
gaagaagaggcacagggctgtgag

SEQ ID NO: 8 - DNA, 28 bp, Homo sapiens
gctggtcctgccgctgcttgtcatggtc

SEQ ID NO: 9 - DNA, 23 bp, Artificial Sequence
  primer
cccagtcacgacgttgtaaaacg

SEQ ID NO: 10 - DNA, 23 bp, Artificial Sequence
  primer
agcggataacaatttcacacagg

(19) United States
(12) Patent Application Publication
(10) Pub. No.: US 2008/0318204 A1
(43) Pub. Date: Dec. 25, 2008

(54) Highly-Sensitive Genomic Assays Employing Chimeric Bacteriophage Standards

(76) Inventors: Linqi Zhang, New York, NY (US); David D. Ho, Chappaqua, NY (US)

Correspondence Address: KLAUBER & JACKSON, 411 HACKENSACK AVENUE, HACKENSACK, NJ, 07601, US

(21) Appl. No.: 10/497,828

(22) PCT Filed: Dec. 4, 2002
(86) PCT No.: PCT/US2002/038612

§ 371 Date: Nov. 23, 2004

Related U.S. Application Data
(60) Provisional application No. 60/337,930, filed on Dec. 6, 2001.

(51) Int. Cl. C12Q 1/70; C12N 7/00

(52) U.S. Cl. 435/5; 435/235.1

(57) ABSTRACT

Methods are provided for sensitively quantitating at least one pre-selected DNA sequence in a biological sample utilizing hybridization methodology, the method employing as an internal standard an infectious bacteriophage particle comprising a detectable target DNA sequence other than that present in the pre-selected DNA sequence or in DNA quantitated from the biological sample, and as an external standard, an infectious bacteriophage particle comprising at least the pre-selected DNA sequence.

### STATEMENT OF RELATED APPLICATION

The present application claims priority under 35 USC § 119(e) to U.S. Ser. No. 60/337,930 filed 6 Dec. 2001, which application is herein specifically incorporated by reference in its entirety.

### BACKGROUND

For quantifying a genomic target such as a DNA target, an accurate and reliable standard is absolutely necessary. In the instance of DNA standards, most often, plasmid DNA and PCR products are the first choice since they are easy to generate. Measuring optical density (O.D.) of plasmid DNA or PCR products can provide rough estimates of copy number of a standard, by dividing by the molecular weight of the plasmid or PCR products. However, this method has severe defects, largely due to the instability of optical density instruments (spectrometers) in quantifying plasmid and PCR products, which not only varies from laboratory to laboratory but also varies from person to person in the same laboratory. In addition, plasmid DNA and PCR products are prone to instability, as they are found to be sensitive to multiple rounds of freeze-thaw and incidental DNase contamination.

### SUMMARY OF THE INVENTION

In its broadest aspect, the present invention is directed to methods for sensitively quantitating at least one pre-selected DNA sequence in a biological sample utilizing hybridization methodology, the method employing as an internal standard an infectious bacteriophage particle comprising a detectable target DNA sequence other than that present in the pre-selected DNA sequence or in DNA quantitated from the biological sample, and as an external standard, an infectious bacteriophage particle comprising at least the pre-selected DNA sequence.

In the foregoing method, the pre-selected DNA sequence may be part of a viral DNA sequence wherein the presence and amount of a pathogenic virus in a biological sample is desirably detected. Human pathogenic viruses are preferred; HBV or other DNA viruses are most preferred; however, the pre-selected DNA sequence may be of any origin and the sample derived from any organism suspected of harboring the pre-selected DNA sequence. The sample may a bodily fluid such as whole blood, urine, plasma, serum, cerebrospinal fluid, or a biopsy sample containing cells. The DNA detection methodology using a hybridization method preferably may be real-time PCR using molecular beacons or any other forms of probes labeled by florescent dyes. The internal standard may be an infectious bacteriophage engineered to contain a single copy of a detectable sequence. The external standard may be an infectious bacteriophage engineered to contain at least a single copy of the pre-selected DNA sequence that is desirably detected. For the real-time PCR with molecular beacons method, primers and molecular beacons designed to amplify and detect the internal standard sequence, and those designed to amplify and detect the pre-selected DNA sequence in the sample and in the external standard, are employed. The hybridization methodology releases the DNA within the engineered bacteriophages, herein referred to as chimeric bacteriophages, to release the DNA therein.

A preferred but non-limiting bacteriophage that may be used as the internal and external standards by preparing chimeric bacteriophage therefrom which retains infectivity and comprises a single copy of the detectable sequence may be M13, but it is not so limiting. Other bacteriophages, preferably those with single-strand circular DNA, may be used, but it is not so limiting, and double-stranded DNA viruses may be used, such as lambda.

The DNA sequences used for the internal and external standards are engineered into the respective bacteriophage to produce chimeric bacteriophage. Because the inserted sequence does not affect infectivity of the bacteriophage, an absolute quantitation of the amount of target DNA in the standard may be easily assessed by an infectivity assay.

The internal standard chimeric bacteriophage contains a readily-detectable DNA sequence that is not present in the biological sample, such that when a known amount of the internal standard chimeric bacteriophage is added to the sample before processing, the extent of recovery of the internal standard chimeric bacteriophage DNA can be used to assess the recovery of the pre-selected DNA contained therein. Preferably, the pre-selected DNA is from a viral particle, such as a pathogenic virus, in the sample, such that during the processing together of any viral particles in the sample and the added chimeric bacteriophage particles, both undergo the same treatment conditions during sample processing and isolation of DNA, such that the recovery of the internal standard DNA is identically reflective of that of any pre-selected DNA present in the original sample. Although the use of a chimeric bacteriophage of the invention is preferably used to detect viral DNA in a biological sample, it is not so limiting, and it may be used to detect other DNAs in biological sample, such as bacterial, parasite, or even host-derived DNA in a sample.

In a preferred embodiment, the internal standard chimeric bacteriophage contains one copy of a part of the human CCR5 DNA sequence. This internal standard may be used for any assay in which human DNA is not present in the DNA being extracted from the sample. If human DNA may be present, then an internal standard DNA sequence not present in the human genome or detectable by the DNA hybridization methodology in a human DNA sample may be used. In a non-limiting embodiment, the corresponding portion of the human CCR5 gene extending from amino acids 132 to 224 (SEQ ID NO:5) is used. The PCR primers may detect SEQ ID NO:6 and SEQ ID NO:7. A molecular beacon is used which is capable of detecting the aforementioned CCR5 sequence, such as that sequence shown in SEQ ID NO:8. In another example, the internal standard chimeric bacteriophage comprises a portion of the human CD4 DNA sequence, used with corresponding probes and molecular beacon.

In a non-limiting example of the reagents used in the practice of the invention for quantitation of HBV virus in a whole blood sample from a human individual; DNA for quantitation by the method herein is isolated from plasma from the whole blood sample and is essentially free of human DNA. The internal standard is an infectious chimeric M13 phage engineered to contain a single copy of a portion of the human CCR5 DNA sequence such as but not limited to that mentioned above; and the external standard i an infectious chimeric M13 bacteriophage engineered to contain a single copy of a portion of the HBV DNA sequence. The quantitation of the amount of DNA in both of the foregoing chimeric bacteriophage standards is carried out by measuring plaque forming units (PFU); these stable standards may be stored frozen. Primers and molecular beacons designed to amplify and detect the internal standard sequence, and those designed to amplify and detect the pre-selected DNA sequence in the sample and in the external standard, are employed in this non-limiting example, as mentioned above.

The external standard comprising the same detectable pre-selected DNA sequence as is in the sample may be, in the instance where a virus is to be quantitated, a portion of the genome of the virus detectable by the same DNA quantitation method as that of the virus in the sample. Thus, a single copy of the sequence, which the PCR primers and molecular beacon amplify and recognize in the sample, may be engineered into the bacteriophage genome. In a non-limiting example, a bacteriophage particle such as a M13 phage particle for use as a HBV external standard may comprise a single copy of DNA encoding amino acids 127 to 164 of the HBV S gene, the DNA sequence as depicted in SEQ ID NO:1. PCR primers and molecular beacon for amplification and quantitation of this sequence in the external standard, as well as in the sample, are readily preparable. In this instance, primers are prepared which hybridize to SEQ ID NO:2 and SEQ ID NO:3. A useful molecular beacon to detect this sequence recognizes the sequence depicted in SEQ ID NO:4.

As mentioned above, the internal standard may be an infectious bacteriophage engineered to comprise any DNA sequence that is not the pre-selected DNA sequence and is not incidentally present in the sample.

Engineered phage particles comprising the internal standard and external standard sequences provide a highly stable reagent facilely used in performing a highly sensitive assay. As the viability of the phage is unaffected by the insertion of the sequence, an assay for phage PFU provides an accurate quantitation of the number of DNA sequences present in the standard, and thus the standardization of these reagents is simple.

To carry out the method of the invention, in the non-limiting example of detection of HBV in human whole blood, a sample of whole blood is centrifuged and a 100 microliter aliquot of plasma is taken, a known amount of internal standard chimeric CCR5-gene-fragment-containing bacteriophage is added, and the DNA extracted. Real-time PCR for HBV and the CCR5 fragment is performed on the processed sample, along with a HBV external standard using the chimeric bacteriophage containing the portion of the HBV sequence detected by the same primers and molecular beacon used for the sample. The recovery of CCR5 in the sample and the amount of HBV detected is used to calculate the actual amount of HBV in the original sample.

The invention is also drawn to phage particles comprising the inserted internal standard sequence or external standard sequence, particularly wherein such insertions do not have a deleterious effect on the viability of the virus and thus accurate quantitation of the number of copies of the particular DNA in a sample of the virus. Thus, the number of PFU of a sample is equal to number of internal standard sequences or external standard sequences present in the phage standard. In non-limiting examples, an M13 phage with SEQ ID NO:1 inserted at position 6247 is embraced herein, as is a M13 phage particle with SEQ ID NO:5 inserted at position 6247. These are merely exemplary of the engineered phages of the invention.

These and other aspects of the invention will be appreciated from the following brief descriptions of the figures and ensuing detailed description.

### BRIEF DESCRIPTION OF THE FIGURES

FIG. 1 depicts an example of the method of the invention for accurately quantitating HBV genomes in a biological sample in which an internal standard of Phage-CCR5 is added to the sample before DNA extraction and real-time PCR for HBV, and comparison to a standard curve derived from an external standard using Phage-HBV.

FIG. 2 shows a molecular beacon for the detection of a portion of the HBV genome (A), and a schematic (B) showing the hybridization of the beacons to the target sequences, resulting in separation of the fluorophore and quencher at the ends of the beacon and consequent fluorescence.

FIG. 3A-C shows a schematic of the PCR amplification of DNA containing a target sequence for the beacon (FIG. 3A), and a standard curve derived from increasing amounts of Phage-HBV added to samples (FIGS. 3B-C).

FIGS. 4A-B shows the genomic locations of the primers and beacon used in a HBV assay of the invention. The shaded sequences at the 5′ and 3′ ends of the sequence encoding amino acids 127-164 are the PCR primers, and the darker, centrally-located sequence that of the recognition sequence of the beacon.

FIG. 5 shows the locations of primers and a beacon used in the CCR5 assay. The shaded sequences at the 5′ and 3′ ends of the portion of the CCR5 gene are the PCR primers, and the darker, centrally-located sequence that of the recognition sequence of the beacon.

FIGS. 6A-B shows two examples of the sensitivity and dynamic range of a HBV assay of the invention.

FIGS. 7A-F depict the stability of Phage comprising a HBV polynucleotide after storage for 3-4 weeks at 4° C. (FIGS. 7A-B), room temperature (FIGS. 7C-D), and 37° C. (FIGS. 7E-F).

FIGS. 8A-D show the concurrent (multiplex) assays for both the HBV sequence (FIGS. 8A-B) and CCR5 (FIGS. 8C-D) sequence in a sample and that there is no interference between HBV and CCR5 amplification in the same tube.

### DETAILED DESCRIPTION OF THE INVENTION

Before the present methods are described, it is to be understood that this invention is not limited to particular methods, and experimental conditions described, as such methods and conditions may vary. It is also to be understood that the terminology used herein is for the purpose of describing particular embodiments only, and is not intended to be limiting, since the scope of the present invention will be limited only the appended claims.

As used in this specification and the appended claims, the singular forms “a”, “an”, and “the” include plural references unless the context clearly dictates otherwise. Thus for example, references to “a method” includes one or more methods, and/or steps of the type described herein and/or which will become apparent to those persons skilled in the art upon reading this disclosure and so forth.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to describe the methods and/or materials in connection with which the publications arc cited.

The assays of the invention provide highly accurate and sensitive means for quantitating the level of a preselected DNA sequence in a sample. Preferably suited for detecting the number of viral particles in a biological sample but not being so limited, the assays employ standards which are viable bacteriophage particles comprising the appropriate DNA sequence: for an internal standard, where recovery of input DNA is assessed and the resultant detected level corrected thereby, utilizes bacteriophages containing a DNA sequence entirely foreign to the input DNA, such that the detectability of the internal standard is not affected by any components from the sample or assay. The external standard, used to generate a standard curve or single-point calibrator, is a viable bacteriophage particle comprising at least the same DNA sequence that is detected in the sample, such that the reagents for quantitation of the pre-selected DNA in the sample are used for the external standard. Using a hybridization-based DNA detection assay, the DNA in the standard bacteriophages added to the assays are released from the bacteriophage at the first melting cycle.

The genomic assay of the invention utilizing viable phages comprising external and internal standard DNA sequences offers a highly accurate and sensitive assay for several reasons. First, the phage particles are easy to generate (approximately 10⁹ PFU/ul). Secondly, the phages and therefore the DNA therein the bacteriophages are easy to quantify, by measuring PFU, which matches with that measured by limiting dilution PCR. Thirdly, it is easy to maintain and transfer the phage particles because of their resistance to DNase treatment and temperature changes. And lastly, it is easy to be precise because no DNA extraction is needed: the PCR conditions release the standards' DNAs from their bacteriophage particle packages. In the case of M13, the single-strand, circular form of DNA is automatically released into the PCR reaction mixture once heated to 95 C, during the initial segment of template denaturation. The engineered phage particles of the invention are referred to herein as chimeric phages, to reflect the presence non-phage DNA within the phage genome.

As will be seen in the examples below, the assay of the present invention tailored for the detection of HBV has a 6-log dynamic range, and can detect as little as 10 copies of HBV up to 10,000,000 copies. In contrast, the Roche HBV Monitor assay has a sensitivity of 200 copies, operates over 3 logs and thus can detect 200 to 200,000 copies Bayer's HBV bDNA assay operates over 4 Meq and is sensitive to 0.7 mEq (×10⁶) and thus detects from 0.7 to 5,000 Meq (×10⁶).

The chimeric phage are prepared following standard recombinant DNA techniques. In brief, target sequences are amplified by PCR and inserted into the SmaI or XmaI site by overnight ligation using T4 ligase (Gibco). Since insertion of a DNA fragment into the SmaI or XmaI site will disrupt the alpha-peptide sequence, the loss of beta-galactosidase activity is therefore expected which is reflected by white instead of blue plaques. By picking multiple white plaques followed by a series of sequencing characterization, we can therefore select those M13 phages carrying the desirable target sequences. The sequence of the M13 phages is 7250 bp long and its full sequences and restriction endonuclease information can be found on the Internet at www.lifetech.com. The target sequences herein are invariable and are inserted into the SmaI or XmaI site in the multiple cloning site.

In addition to the target sequences mentioned above, one can insert any DNA sequences that are identical to the genome sequence to be detected into the M13 bacteriophage. To avoid the detection of contaminated genomic DNA in the test sample, however, one can always insert cDNA sequences into the M13 phage from which no genomic DNA will be amplified due to a large intron that exists between the different exons. Thus, in an example of this embodiment of the invention, primers and beacons for the human CD4 gene have been prepared in accordance with the teachings herein. Moreover, there are many single and double stranded DNA bacteriophage can be used in the same format for quantitative standard. In the case of single-stranded DNA, M13 is undoubtedly the most convenient and reliable choice much knowledge about this vector and its biology is available. As for the double stranded bacteriophage, the lambda series are a preferred choice for the same reasons as above. All of these recombinant phages are extremely easy to produce, purify and quantify by measuring PFU.

### EXAMPLES

The following example is put forth so as to provide those of ordinary skill in the art with a complete disclosure and description of how to make and use the methods and compositions of the invention, and are not intended to limit the scope of what the inventors regard as their invention. Efforts have been made to ensure accuracy with respect to numbers used (e.g., amounts, temperature, etc.) but some experimental errors and deviations should be accounted for. Unless indicated otherwise, parts are parts by weight, molecular weight is average molecular weight, temperature is in degrees Centigrade, and pressure is at or near atmospheric.

M13 bacteriophage DNA standards were made as follows: the amplicon of interest was generated using the appropriate primers for the assay and a Pfu polymerase to generate a blunt ended product. The product was purified on a 1% agarose gel and ligated into M13mp 18 RF DNA (Gibco) according to manufacturer's instructions. The ligation product was used to transform DHα5F′ competent cells (Gibco). Plaques generated from phage containing inserts were identified using blue/white selection for the absence of β-galactosidase activity. Positive plaques were screened by PCR using primers M13-pUC-f(5′-CCCAGTCACGACGTTGTAAAACG-3′)(SEQ ID NO:9) and M13/pUC-b (5′-AGCGGATAACAATTTCACACAGG-3′) (SEQ ID NO:10) in a 30 cycle PCR (95° C. for 30 s, 55° C. for 30 s, 72° C. for 1 m). These are the generic primers for M13 phage flanking the region of insertion. They can therefore be used to screen whether the phage has any insert or not. Fragments of the correct size were further screened by sequence analysis. Bacteriophage was tittered and serial dilutions were made in RNAse-free water. Bacteriophage was put directly into the PCR reaction, as the 10 minute 95° C. denaturation step was sufficient to expose the phage DNA.

An external standard curve was generated for each real-time PCR assay using a minimum of 6 replicates ranging from 2.5×10⁶ to 2.5×10¹. As the phage is single stranded, one particle corresponds to 0.5 double-stranded DNA copies in the real-time PCR assay. Phage standards were stable at room temperature and 4° C., although stocks were maintained at −20° C.

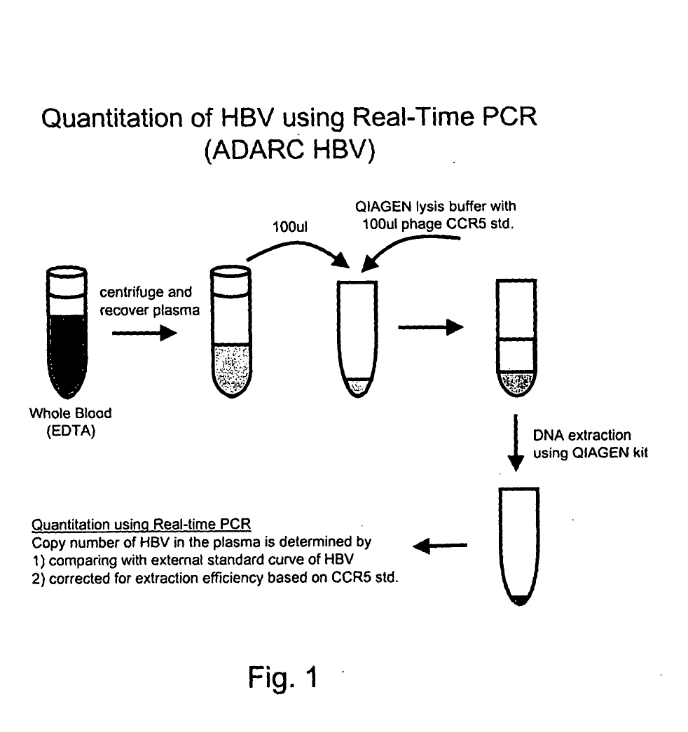

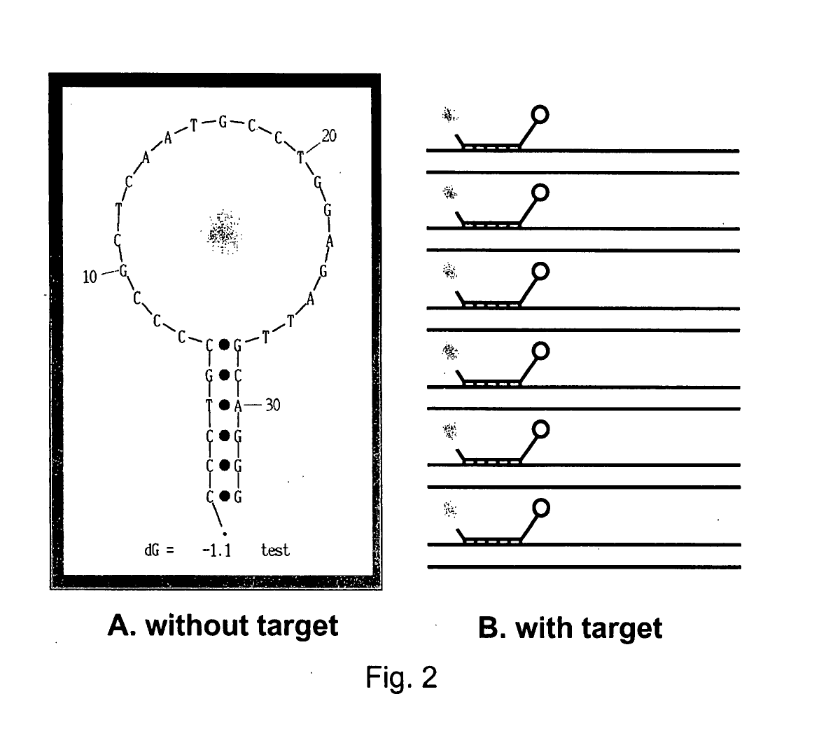

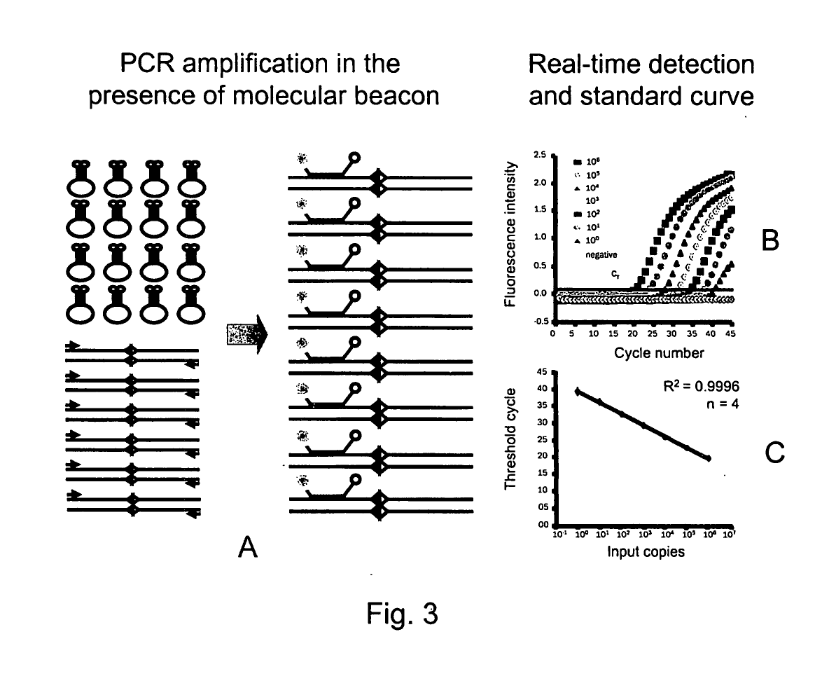

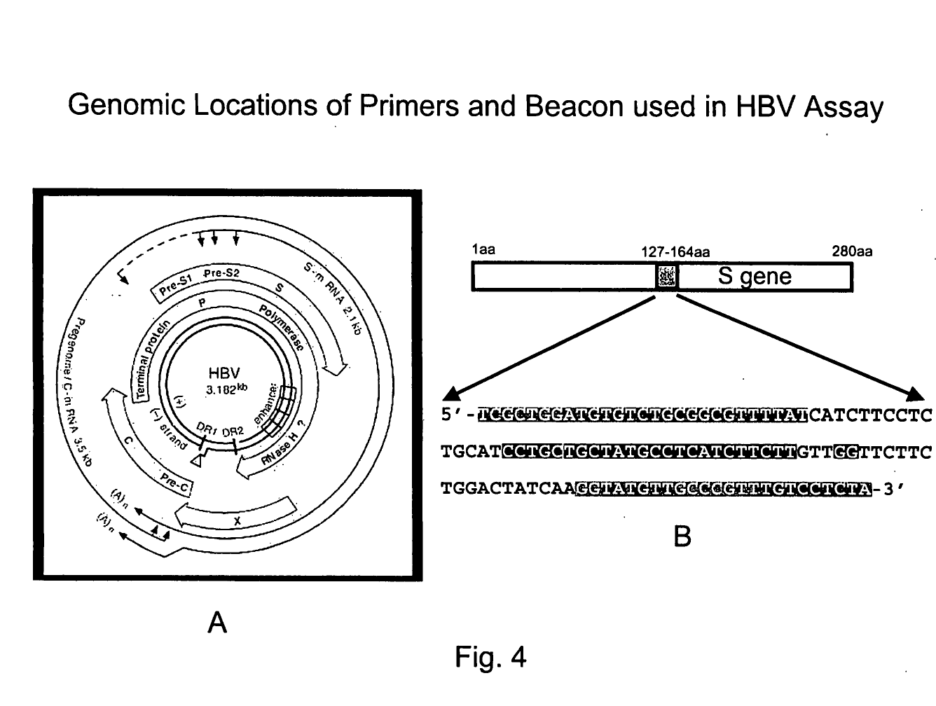

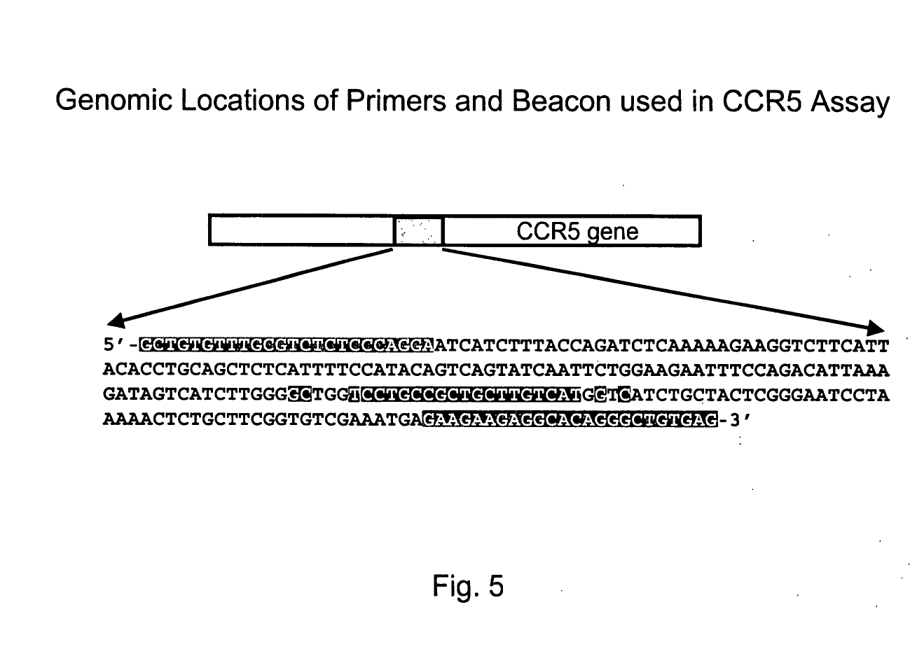

The method of the invention is shown in FIG. 1, for accurately quantitating HBV genomes in a biological sample in which an internal standard of Phage-CCR5 is added to the sample before DNA extraction and real-time PCR for HBV, and comparison to a standard curve derived from an external standard using Phage-HBV. FIG. 2 shows a molecular beacon for the detection of a portion of the HBV genome (A), and a schematic (B) showing the hybridization of the beacons to the target sequences, resulting in separation of the fluorophore and quencher at the ends of the beacon and consequent fluorescence. FIG. 3 shows a schematic of the PCR amplification of DNA containing a target sequence for the beacon, and a standard curve derived from increasing amounts of Phage-HBV added to samples. FIG. 4 shows the genomic locations of the primers and beacon used in a HBV assay of the invention. The shaded sequences at the 5′ and 3′ ends of the sequence encoding amino acids 127-164 are the PCR primers, and the darker, centrally-located sequence that of the recognition sequence of the beacon. FIG. 5 shows the locations of primers and a beacon used in the CCR5 assay. The shaded sequences at the 5′ and 3′ ends of the portion of the CCR5 gene are the PCR primers, and the darker, centrally-located sequence that of the recognition sequence of the beacon.

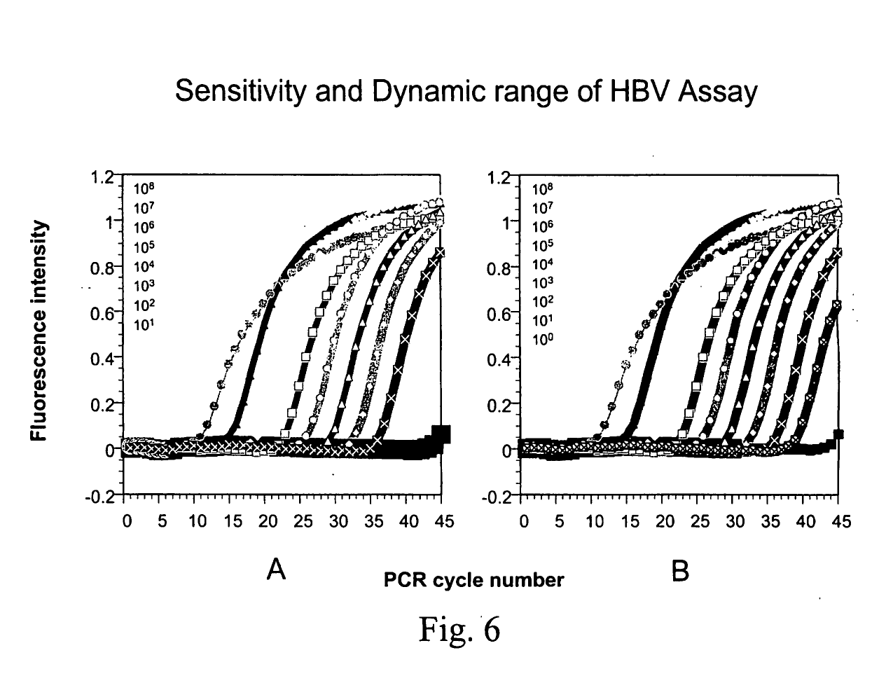

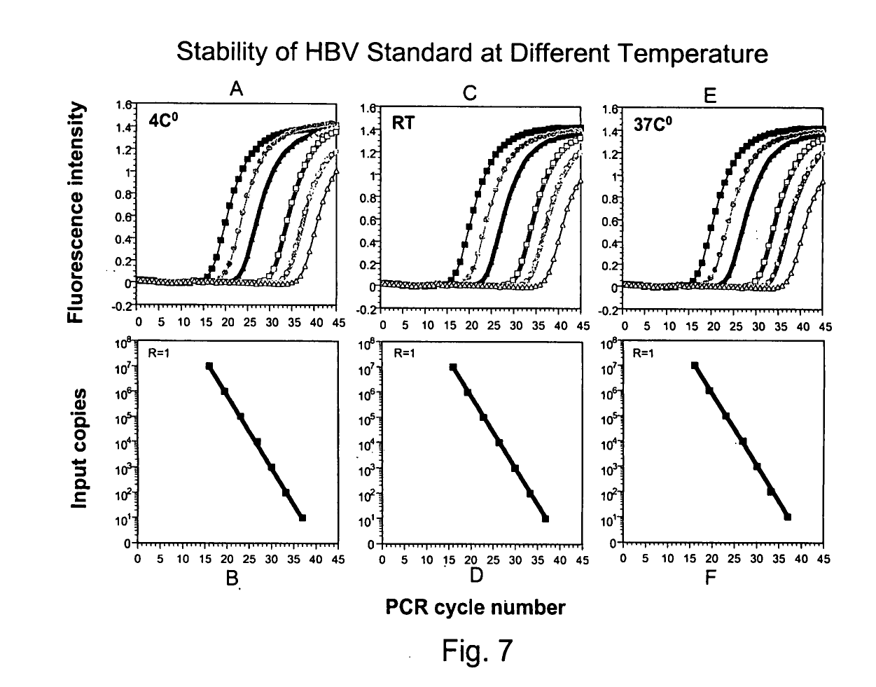

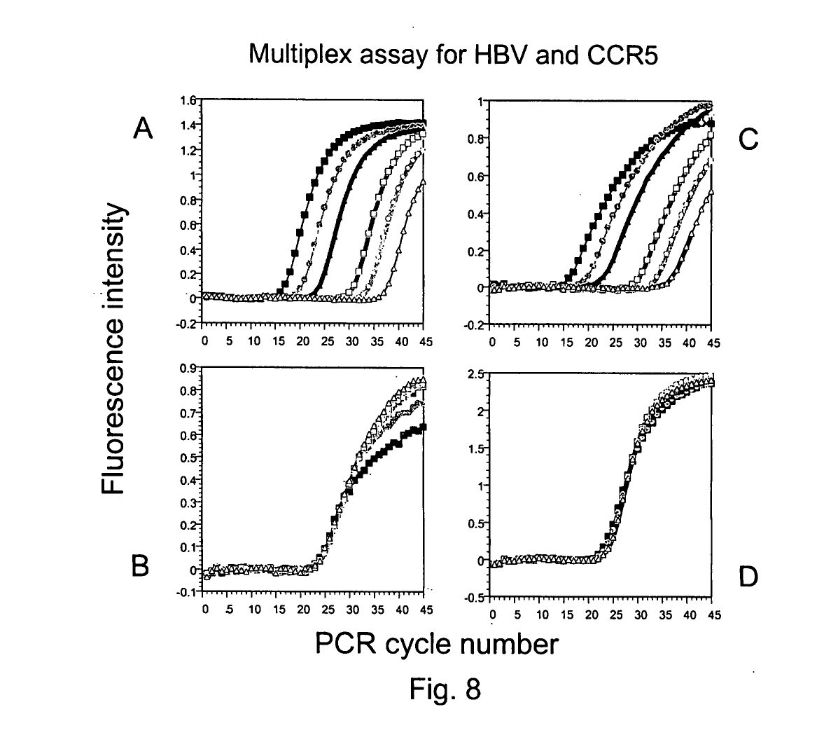

FIG. 6 shows two examples of the sensitivity and dynamic range of a HBV assay of the invention. FIG. 7 depicts the stability of Phage comprising a HBV polynucleotide after storage for 3-4 weeks at 4 C, room temperature, and 37° C. FIG. 8 shows the concurrent (multiplex) assays for both the HBV sequence and CCR5 sequence in a sample and that there is no interference between HBV and CCR5 amplification in the same tube. The results show that the generation of M13 phage comprising a HBV gene, or a CCR5 gene is extremely efficient and the titer of infectious phages is as high as 10⁹ per microliter culture supernatant. Further, the method of the invention achieves a very high correlation between plaque-forming units of the infectious M13 phage comprising either a HBV gene or a CCR5 gene and the copies numbers measured by limiting dilution quantitative PCR.

What is claimed is:

1. A method for quantitating at least one pre-selected DNA sequence in a biological sample utilizing hybridization methodology, wherein the method employs as an internal standard an infectious M13 bacteriophage particle comprising a detectable target DNA sequence other than that present in the pre-selected DNA sequence or in DNA quantitated from the biological sample, and as an external standard, an infectious M13 bacteriophage particle comprising the pre-selected DNA sequence.

2. The method of claim 1, wherein the hybridization methodology is real-time PCR amplification using molecular beacons.

3. The method of claim 1, wherein the internal standard is an infectious M13 bacteriophage comprising a portion of a human CCR5 gene.

4. The method of claim 3, wherein the portion of said human CCR5 gene is SEQ ID NO:5.

5. The method of claim 1, wherein the pre-selected DNA sequence is a portion of a hepatitis B virus.

6. The method of claim 1, wherein the external standard is an infectious M13 particle comprising a portion of a hepatitis B virus genome.

7. The method of claim 5, wherein the external standard is an infectious M13 particle comprising a portion of a hepatitis B virus genome.

8. The method of claim 7, wherein the portion of the hepatitis B genome is a portion of a hepatitis B S protein.

9. The method of claim 8, wherein the portion is SEQ ID NO:1.

10. The method of claim 1, wherein the sample is selected from a group consisting of whole blood, urine, plasma, serum, cerebrospinal fluid, or a biopsy sample containing cells.

11. An infectious chimeric M13 bacteriophage comprising SEQ ID NO: 1.

12. An infectious chimeric M13 bacteriophage comprising SEQ ID NO:5.
